(19) United States
(12) Patent Application Publication
(10) Pub. No.: US 2024/0350107 A1
(43) Pub. Date: Oct. 24, 2024

(54) SYSTEMS AND METHODS FOR MEGAVOLTAGE X-RAY INDUCED POSITRON EMISSION (MVIPE) IMAGING

(71) Applicants: The Brigham and Women's Hospital, Inc., Boston, MA (US); University of Massachusetts Lowell, Boston, MA (US)

(72) Inventors: Piotr Zygmanski, West Roxbury, MA (US); Davide Brivio, Boston, MA (US); Erno Sajo, Lowell, MA (US)

(21) Appl. No.: 18/291,451

(22) PCT Filed: Jul. 25, 2022
(86) PCT No.: PCT/US22/38206

§ 371 Date: Jan. 23, 2024

Related U.S. Application Data
(60) Provisional application No. 63/225,415, filed on Jul. 23, 2021.

(51) Int. Cl. A61B 6/00; A61B 6/42

(52) U.S. Cl. CPC A61B 6/5235; A61B 6/4241; A61B 6/4258; A61B 6/4266

(57) ABSTRACT

Systems and methods for detecting and imaging high Z agents within anatomy of a subject are provided. A fast photon counting detector array is positioned relative to the subject to detect coincident photons having 511 keV energy and opposing directions. The trajectories of those photons can be determined, thereby leading to the origin of the photons, namely, the location of an annihilation event. Based on the number of these events, the high Z agent can be detected and/or imaged within the anatomy, thereby providing useful information for clinicians regarding the distribution of high Z agents in the anatomy and/or the behavior of the therapeutic medical high energy photon source.

### CROSS-REFERENCE TO RELATED APPLICATIONS

The present application claims priority to U.S. Provisional Patent Application No. 63/225,415 that was filed Jul. 23, 2021, the entire contents of which are hereby incorporated by reference.

### STATEMENT REGARDING FEDERALLY SPONSORED RESEARCH

Not applicable.

### BACKGROUND OF THE INVENTION

The disclosed technology relates to systems, methods and devices for mapping of interaction of megavoltage ionizing radiation with tissue and/or with radiosensitization/contrast agent (material) (RCA) absorbed by the tissue. In particular, the present disclosure is directed to systems, methods and devices for efficient detection and mapping of distribution of radiosensitization/contrast agent and/or absorbed dose in tissue.

In treatment of cancer it is important to maximize delivery of ionizing radiation energy to the cancer cells and minimize the delivery to normal cells. This can be achieved by multiple strategies including: (A) imaging of patient anatomy and accurately setting up patient for treatment, (B) monitoring or imaging changes of location and deformation of anatomical structures being irradiated (C) monitoring or imaging of dynamic linear accelerator (linac) output and where within the anatomy dose is delivered (D) sensitization of cells with materials that differentially increase toxicity or ionization capability of radiation in cancer vs normal cells, and finally (E) imaging the biodistribution of the radiosensitization/contrast agent for the safety and assessment of the radiobiological effect. So far detection of dose inside patient (C) has not been possible without implanting or inserting sensors, which is impractical, inefficient and requires a surgical procedure or by using computational methods based on measured of x-ray flux generated by linac and detected outside of the patient. Second, determination of concentration of radiosensitization/contrast agent (E) using Computed Tomography and X-ray Fluorescence CT has faced multiple problems due to small concentrations of the agent and attenuation of the useful signal by the patient body, which result in poor detectability and/or very long scanning times and large dose from imaging. Determination of biodistribution of dose and/or radiosensitization/contrast agent in vivo is important not only for the established Intensity Modulated Radiotherapy (IMRT) or Volumetric Modulated Arc Therapy (VMAT) but also for new or under development radiotherapy modalities such as FLASH RT, micro- or mini-beam RT. For instance, in FLASH RT the dose rates are much higher than in the standard RT and in micro-mini-beam the doses are very heterogenous requiring new technologies to generate, control and monitor (image) the radiation beams.

Given the above there is a need for new systems and methods for detection of distribution of ionizing energy deposited inside patient body (C) and/or distribution of radiosensitization/contrast agent within patient anatomy (E) that are more efficient, practical and sensitive.

### BRIEF SUMMARY OF THE INVENTION

In an aspect, the present disclosure provides a system for detecting and imaging high Z agents within anatomy of a subject. The system includes a therapeutic medical high energy photon source, a fast photon counting detector array, a processor, and a memory. The source is adapted to provide megavoltage ionizing radiation with an emitted photon energy of no lower than 1.02 MeV. The array is positioned so that at least two pixels are oriented to simultaneously measure two coincident, 511 keV photons with opposing directions originating from a location. The memory has stored thereon instructions that, when executed by the processor, cause the processor to: a) initiate delivery of the megavoltage ionizing radiation from the therapeutic medical high energy photon source to the location; b) initiate detection of the two coincident photons with opposing directions originating from the location; c) determine photon trajectories of the two coincident photons with opposing directions, thereby providing a source location for the two coincident photons where the photon trajectories meet; and d) generate a report including a high Z agent position within the anatomy of the patient derived from the trajectories of the two coincident photons with opposing directions within the patient's anatomy. The high Z agent has an atomic number of greater than 7.4 and/or a density that is greater than surrounding tissue within the anatomy.

In another aspect, the present disclosure provides a method of detecting and imaging high Z agents within anatomy of a subject. The method includes: a) delivering megavoltage ionizing radiation from a therapeutic medical high energy photon source to at least one high Z agent located within the anatomy of the subject; b) subsequently, detecting two coincident photons with opposing directions originating from the high Z agent with a fast photon counting detector array positioned so that at least two pixels are oriented to simultaneously measure the coincident photons with opposing directions; c) determining photon trajectories of the detected two coincident photons, thereby providing a source location for the two coincident photons where the photon trajectories meet; and d) generating a report including a high Z agent position within the anatomy of the subject derived from the trajectories of the two coincident photons. The high Z agent has an atomic number of greater than 7.4 and/or a density that is greater than surrounding tissue within the anatomy.

Any citations to publications, patents, or patent applications herein are incorporated by reference in their entirety. Any numerals used in this application with or without about/approximately are meant to cover any normal fluctuations appreciated by one of ordinary skill in the relevant art.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1 is a schematic interaction of megavoltage x-rays [2] generated by radiotherapy linear accelerator [1] during patient treatment, which x-rays [2] undergo a variety of interactions with patient/tissues [3] and RCA [4,5] giving rise to deposited ionizing energy (dose) [6] and radiosensitization [4] and/or imaging contrast [5]. Megavoltage x-ray interactions with tissue [3]/RCA [4,5] include: photelectric/fluorescence [7,8], Compton scatter [9], pair-production [10]. Direct x-rays [11] emerge without any interaction. Electron-pair production [10]results in generation of two positron annihilation gammas [12], which are detected in coincidence by opposing detector array elements [14]. The detection system [14] performs spatio-angular-temporal analysis [15], discrimination of contributions [16], reconstruction of dose [17] and/or RCA biodistribution in the tissue [18], and correlation with anatomy determined utilizing computed tomography [19] and/or magnetic resonance imaging obtained before and/or during treatment.

FIG. 2 is a schematic distinction between annihilation gammas [13] originating from RCA-free tissues vs from tissues with RCA [4,5]. Experimental determination of biodistribution of dose [6] and/or RCA [4.5] employing of MeV-X-ray-Induced-Positron-Emission (MVIPE) [2-10-12] and detection of annihilation gammas [13] in coincidence [14] is the subject of this invention.

FIG. 3 is a schematic diagram of potential regions occupied by MVIPE detector array.

FIG. 4 shows cross-sections of the photoelectric effect (PE) and pair production (PP) in the nuclear field in water, Au and in a mixture Au-water with 10% of Au by weight (10 wt %) as function of incident x-ray energy.

FIG. 5 shows Deterministic 1D simulations of photon flux after Compton background correction (Δflux) as function of depth in a water phantom with various amounts of AuNP exposed to different photon sources. Panel (A) shows 511 keV A flux with 0 wt % (water), 1% wt and 10 wt % AuNP; the 1% wt AuNP is almost undistinguishable from the water; Panel (B) shows k_{α1}Δ flux, with 0.1 wt %, 1 wt % and 10 wt % AuNP; Panel (C) shows 511 keV Δ flux generated by AuNP only, obtained from the results in (A) by subtracting the 511 keV photons generated in water (0 wt % AuNP). Data are normalized to one incident source photon.

FIG. 6 shows 3D MC simulation: Δflux at each detector plotted as a function of detector angle with respect to the incident beam direction (0°). Panel (A) shows 511 keV photons generated by a 15MVsource for 0 wt % AuNP (red) and 10 wt % AuNP; Cyan and blue are the respective 0 wt % and 10 wt % cases but water in the large cylindrical phantom is replaced by air. Panel (B) shows k_{α1} photons generated by 150 kVp source interactions with 10 wt % AuNP concentration using water and air filling the large cylinder. Panel (C) shows 511 keV photons from AuNP only, after subtraction of the contribution from water. Uncertainties include MC and error propagation after Compton background.

FIG. 7: Deterministic 1D simulations of dose deposited as a function of depth in a water phantom with and without a 1 wt % AuNP region (dashed and solid lines, respectively) using MV (shown in Panel A) sources and kVp (shown in panel B) sources. Dose is reported in cGy normalized by the number of source photons incident on 1 cm² phantom surface.

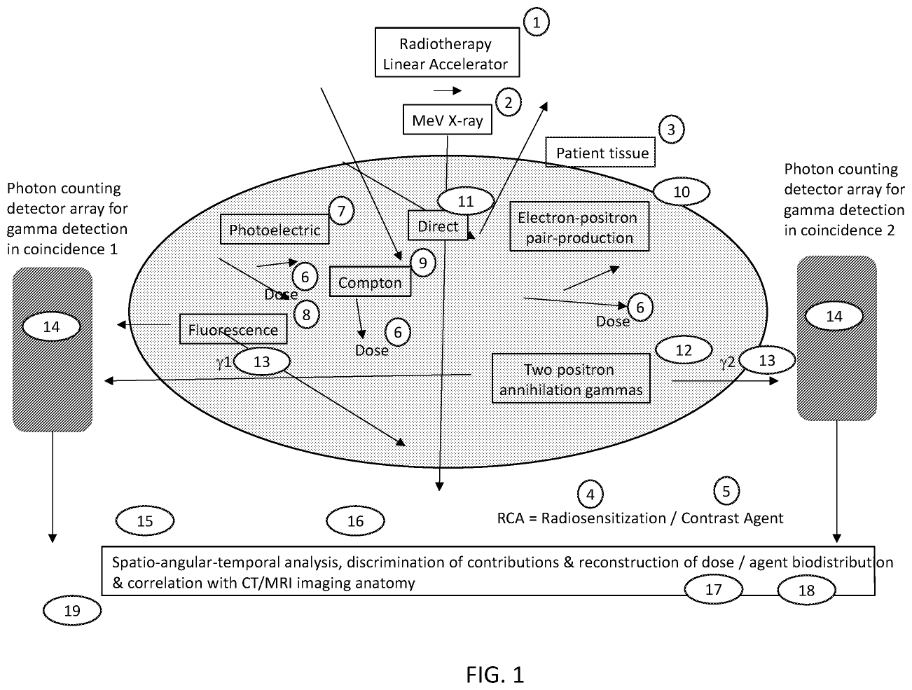

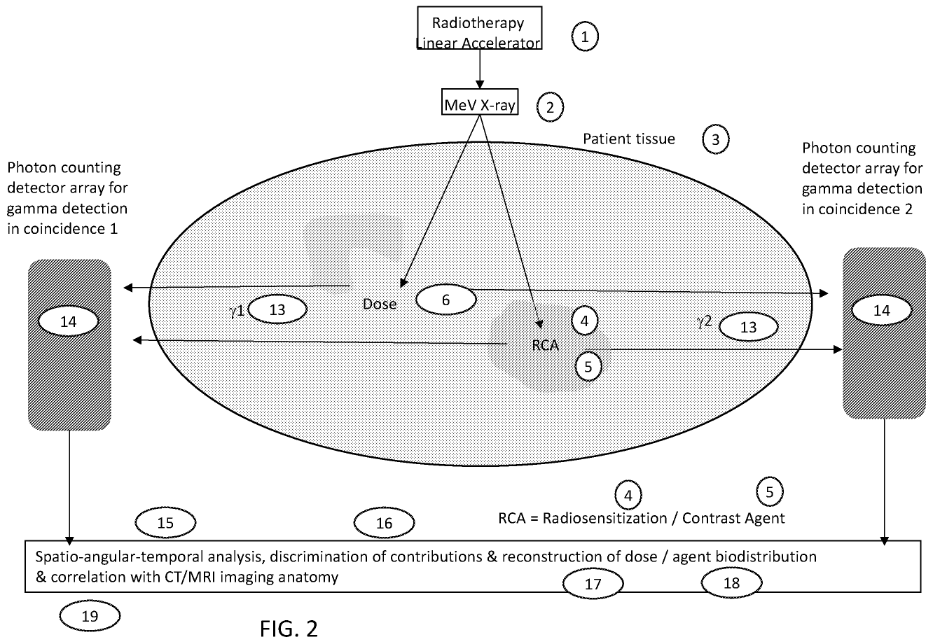

### DETAILED DESCRIPTION

The present disclosure provides detection method and system that overcome the aforementioned drawbacks by introducing a novel approach consisting in detection of two photons in coincidence arising from interaction of megavoltage x-rays with the patient/RCA.

In one aspect of the disclosure, radiosensitization/contrast agent (RCA) is a material introduced into patient body by means of intravenous, oral, interstitial, gynecological, urological, or pulmonary (inhalation) means to either increase contrast of specific tissues vs other tissues for improved radiological imaging and intervention and/or to sensitize tumor cells to the radiotherapy x-rays.

In another aspect of the disclosure, megavoltage x-ray radiation interacting with cancer and normal cells and/or with RCA inside patient leads to production of positrons-electron pairs (via so called pair production near nuclei), subsequently positron undergoes annihilation with one of the electrons in the medium, which results in emission of two photons traveling in opposing directions. These two photons are detected in coincidence to give rise to photon counting detector signal and reconstruction of the trajectory and location of the origin of positron annihilation. From the number of such origins of positron annihilation the distribution of energy deposited in tissue and RCA is obtained.

The foregoing and other aspects and advantages of the invention will appear from the following description. In the description, reference is made to the accompanying drawings that form a part hereof, and in which there is shown by way of illustration a preferred embodiment of the invention. Such embodiment does not necessarily represent the full scope of the invention, however, and reference is made therefore to the claims and herein for interpreting the scope of the invention.

Previous methods of determination of x-ray radiation inside patient during treatment rely on several types of methods. The first one is a direct measurement with point-detectors inserted in cavities or on skin or implanted by surgical procedure. Only a single point of measurement can be obtained with a single detector and detector must be small and mechanically compatible/biocompatible with the tissue. Another type of method relies on measuring of megavoltage linac beam externally of the patient, either upstream or downstream, by using a 2D detector array (diode, ion chamber) or EPID, and reconstructing dose inside patient using various algorithms. This method is a computational method based on transmitted flux information which depends on the modulation of the beam by the linac and total absorption by the patient body, but it does not measure dose deposition signals originating from the patient body itself. Another method is based on Cherenkov light radiation produced by charge particles (electrons) travelling in the tissue as a result of x-ray interaction with patient. This method is limited by the penetration of blue light through the tissue and thus limited to cutaneous tissues or patient surface. Another method is based on detection of acoustic wave generated by x-rays interacting with tissue by using ultrasound detector arrays. This method is still under development and little is known about its limitations and potential except for the necessity for the ultrasound detectors to be in physical contact with patient surface.

Previous methods for determination of distribution of RCA inside patient include either CT or XFCT. CT has very poor contrast and is not capable of detection of practical amounts of RCA inside tissues without using a lot of radiation to image the patient. XFCT is much more sensitive than CT in detection of RCA but is still limited by the small number of x-rays that are absorbed by RCA compared to tissue and attenuation and scatter of the useful fluorescent signal by the subsequent layers of tissue. XFCT is also time-inefficient as it requires step by step scanning different portions of the anatomy with a narrow collimator. Thus far only small objects such as small animals can be scanned using XFCT.

In the present disclosure, the deposited dose inside patient anatomy gives rise to positron annihilation signal, which is measured by external, with respect to patient body, photon counting in coincidence detection system. The measured photon annihilation signal is directly related to the specific anatomy where the megavoltage x-rays deposited the dose. Second, the detection of positron annihilation can be done at lateral directions with respect to the treatment x-ray beamline. Detection of positron annihilation (MeV photons) is also more efficient than detection of fluorescent x-rays (keV photons) due to their larger energy and ability to penetrate thick layers of tissue. Measurement of two annihilation photons in coincidence (matching them in temporal domain) makes it possible to avoid collimation of x-rays emerging from the patient body and improve determination of the origin of the signal inside patient along the line of the two photon trajectories. In XFCT collimation is required, which makes it inefficient technique, and tracing the origin of each fluorescence is not possible.

Disclosed herein is a system and method for detecting and imaging of biodistribution of dose/RCA in patient anatomy. In particular, the disclosed technology may be used to image dose/RCA in a patient with a disease treated by radiotherapy, such as a tumor and other lesions. The imaging system and method rely upon high-energy photons inducing electron-positron pair production, subsequent positron annihilation and generation of two coincident and oppositely traveling 511 keV annihilation photons. The reconstructed trajectories of the photon pair may then be used to detect and generate an image of their source.

The fast photon counting detector array is positioned away from the direct path of the treatment beam entering and exiting patient body (path of primary and Compton scatter radiation) and thus laterally or oblique-laterally with respect to the standard megavoltage x-ray beam-line. The region occupied by the MVIPE detector array is away from patient body but otherwise can be an array of arcs surrounding patient body. In another embodiment the detector array can form a spherical or semi-spherical enclosure with unoccupied areas for x-ray beamline entry/exit. The geometry of detector can be fixed with respect to the rotating x-ray beamline. As used herein, a “subject” may be interchangeable with “patient” or “individual” and means an animal, which may be a human or non-human animal, in need of treatment.

A “subject in need of treatment” may include a subject having a disease, disorder, or condition that is able to be imaged by the technology disclosed herein. For example, a “subject in need of treatment” may include a subject having a cell proliferative disease, disorder, or condition such as cancer (e.g., cancers that form solid tumors such as sarcomas, carcinomas, or lymphomas, including non-small cell lung cancer, colon cancer, cancer of the central nervous system, melanoma, ovarian cancer, renal cancer, prostate cancer, and breast cancer) or other lesions such as vascular or neuronal malformations.

Anatomy is defined as the structure and parts of the body of the patient. Anatomy includes tissues, organs, and organ systems. Tissue is defined as any distinct group of cells that have similar structure and function together as a unit. Examples of tissues include epithelial tissue, connective tissue, muscle tissue, and nervous tissue. Organs are made up of two or more types of tissues organized to serve a particular biological function. Organ systems are defined as groups of organs which work together to carry out a particular biological function. In some embodiments, the imaging technology disclosed herein may be applied to tissues, organs, and organ systems. In other embodiments, the technology disclosed herein may be applied to imaging cell proliferation disease impacting tissues, organs, and organ systems.

As used herein, “high Z” refers to compositions having at least one component with atomic number greater than a certain value or with a density that is greater than surrounding tissue. In some cases, a high Z agent has at least one component with an atomic number of greater than 7.4, including but not limited to, compositions having at least one component with atomic number of greater than 10, greater than 15, greater than 20, greater than 25, greater than 30, greater than 35, greater than 40, greater than 45, greater than 50, greater than 53, greater than 55, or greater than 60. In some specific cases, the high Z agent has at least one component with an atomic number of greater than 53. In some cases, a high Z agent has a density that is greater than surrounding tissue, such as a lung tumor positioned within lung tissue.

In another aspect, image contrast may be accomplished by a high Z agent. In one embodiment, a high Z agent may be a radiosensitization/contrast agent (RCA). In one embodiment the high Z agent and/or the RCA may be gold, platinum, gadolinium, bismuth, tantalum, tungsten, hafnium, silver, or other high atomic number (Z) nanoparticles. In other embodiments, intrinsic biological features may serve as high Z agents due to their composition, density, or ability to perturb a detectable aspect of the imaging modality. For example, lung lesions, bones, or other high Z agents that are present in biological systems, as would be appreciated by a person having ordinary skill in the art.

In one embodiment, a radiosensitization/contrast agent (RCA) may be used to aid imaging. Radiosensitization agents are defined as physical (ionizing dose), chemical (toxicity) or biological (molecular effects) compounds that enhance the effectiveness of radiotherapy in cancer cells. In some embodiments, radiosensitization agents may include traditional chemotherapy agents, such as gemcitabine, interferon-α, 13-cis-retinoic acid, doxorubicin, docetaxel, carboplatin, cisplatin, dactinomycin, methotrexate, 5-FU, bleomycin, and hydroxyurea. In other embodiments radiosensitization agents may include high atomic element (Z) nanomaterials, including bismuth, gold, tungsten, tantalum, hafnium, and silver.

Contrast agents are defined as a substance used to increase the contrast of structures or fluids within patient anatomy during imaging. In radiological imaging, contrast agents may include radiotracers, such as ¹⁸F, as in ¹⁸F-fluorodeoxyglucose (¹⁸F-FDG), ¹¹C, ¹³N, ¹²³I, ⁶⁸Ga, or ⁶⁴Cu. In other embodiments, nanoparticles may also be used as contrast agents. In some embodiments metallic nanoparticles may be used as contrast agents.

In one aspect, a radiosensitization/contrast agent may perform as either a radiosensitization agent or a contrast agent or simultaneously as both a radiosensitization agent and contrast agent.

Administered high Z agents, including RCA and nanoparticles, may be administered as a pharmaceutical composition for delivery via any suitable route. For example, the pharmaceutical composition may be administered via oral, intravenous, intramuscular, subcutaneous, topical, intranasal, and pulmonary route. Examples of pharmaceutical compositions for oral administration include capsules, syrups, concentrates, powders and granules. In some embodiments, the compounds are formulated as a composition for administration orally (e.g., in a solvent such as 5% DMSO in oil such as vegetable oil). In one embodiment, administered high Z agents may have an average concentration in the anatomy being imaged as low as about 0.1 mg/g and as high as about 10 mg/g by weight of RCA vs the tissue. In another embodiment (Gd), administered high Z agents may have an average concentration in the anatomy being imaged as low as about 0.01 nmol/g and as high as about 100 nmol/g of RCA vs the tissue.

The technology disclosed herein utilizes a therapeutic medical high energy photon source. In one embodiment the high energy photon source may be an x-ray radiation source. In one embodiment, the therapeutic medical high energy photon source emits photons with sufficient energy to induce electron-positron pair production in the irradiated material, such as the patient's anatomy. In one embodiment the emitted photons have an energy greater than 1.02 MeV. In some embodiments, the photon beam energy may be 1 MV, 2 MV, 3 MV, 4 MV, 5 MV, 6 MV, 7 MV, 8 MV, 9 MV, 10 MV, 11 MV, 12 MV, 13 MV, 14 MV, 15 MV, 16 MV, 17 MV, 18 MV, 19 MV, 20 MV, 21 MV, 22 MV, 23 MV, 24 MV, 25 MV, 26 MV, 27 MV, 28 MV, 29 MV, or 30 MV. In some embodiments the high energy photon source is a radiotherapy beam. In some embodiments, the x-ray radiation source may be a linear accelerator (linac), Co-60, Van de Graaff generator, or betatron. In some embodiments, the source may be operated with an open, closed, or partially open multi-leaf collimator (MLC).

In one embodiment, the therapeutic medical high energy photon source used on a patient in need of treatment by radiotherapy may be the same therapeutic medical high energy photon source used in the disclosed imaging system and method.

In one aspect, the lower energy limit of the therapeutic medical high energy photon source is determined by the minimum photon energy capable of inducing electron-positron pair production. Without being limited as to theory, the incoming photon must have an equal-to-or-greater-than energy than the rest mass of the electron (0.511 MeV). As such, in one embodiment, the lower energy limit of the therapeutic medical high energy photon source may be 1.022 MeV.

The subsequent annihilation of electron-positron pairs generated by the higher-energy photon source generates two coincident, correlated 511 keV photons with opposite direction and orthogonal polarization.

Detection of the two coincident 511 keV photons with nearly opposite direction may be accomplished with a photon counting array detector that has at least two pixels oriented to simultaneously measure the two coincident 511 keV photons. In one embodiment, the two pixels would be positioned approximately equal distance away from the patient anatomy at a relative angle of 180° to one another. In another embodiment, greater than two pixels may be use. In yet another embodiment, the photon counting array detector would form an array that surrounds the patient in a ring structure, with the patient anatomy located at a central point on the ring.

In one embodiment, the detection of 511 keV photons utilizes energy discrimination detectors to distinguish them from Compton-scattered radiation. In another embodiment, the detection of the 511 keV photons utilizes coincidence counting of the photon pairs. In yet another embodiment, the fast photon counting detector array discriminates between Compton scattering photon and positron annihilation photons by their temporal, spatial, angular, or spectral distribution. The algorithm has to take into account the full knowledge of directions, spatial location, and temporal coincidence and model different sources and reconstruct their quantities in an iterative fashion. Another embodiment is a twofold measurement—before application of RCA and after application. With these two datasets (reference or background vs present) one can subtract the former.

The algorithm has to take into account the full knowledge of directions (angles), spatial locations, and temporal coincidence. One known method for taking into account the full knowledge of directions, spatial locations, and temporal coincidence is the body of algorithms used in Pet imaging or combined PET-Compton camera imaging. The algorithm further has to model the biodistribution of RCA and/or dose and reconstruct their quantities in an iterative fashion. Iterative reconstructions use the standard regularization techniques such as Tikhonov, Bayesian, Total Variation, Compressed Sensing, and other machine learning and optimal control/dynamic systems. Skilled artisans will understand how the concepts of mathematical regularization and total variation denoising are applied to this disclosure.

In one embodiment, the photon counting array detector is a Compton detector, such as the one described by Peng, et al. 2019, wherein the kinematics of the Compton scattering is used to deduce the initial photon trajectory and the source region. In another embodiment, the angular and temporal correlation of photons using Compton/coincidence detector may be used to discriminate between Compton scattering and true annihilation 511 keV photons pairs.

In one aspect of the disclosed technology, image reconstruction may be performed akin to Positron Emission Tomography (PET) imaging wherein coincidence detection of the 511 keV photon pairs generated through positron annihilation eliminates the need for pin-hole collimators. In one embodiment, the detection of coincident 511 keV photon pairs may be performed without collimation or with partial collimation.

Disclosed herein is a method of detecting and imaging high Z agents within anatomy of a subject. The method involves four steps: a) delivering megavoltage ionizing radiation from a therapeutic medical high energy photon source to at least one high Z agent located within the anatomy of the subject; b) subsequently, detecting two coincident photons with opposing directions originating from the high Z agent with a fast photon counting detector array positioned so that at least two pixels are oriented to simultaneously measure the coincident photons with opposing directions; c) determining photon trajectories of the detected two coincident photons, thereby providing a source location for the two coincident photons where the photon trajectories meet; and d) generating a report including a high Z agent position within the anatomy of the subject derived from the trajectories of the two coincident photons.

In one aspect, the system and method disclosed herein is controlled by a processor with memory capable of initiating the various method steps disclosed herein. A person skilled in the art will recognize that a processor with memory is common to computed tomography techniques, such as PET, are already well documented in the art, and are not the focus of the present disclosure.

In another aspect, a 3-dimensional image of the patient's anatomy may be generated by sequentially performing aforementioned method steps at various known orientations and locations relative to the patient's anatomy. In one embodiment, the patient would remain in one orientation and the source and detectors would move around the patient.

### Miscellaneous

Unless otherwise specified or indicated by context, the terms “a”, “an”, and “the” mean “one or more.” For example, “a molecule” should be interpreted to mean “one or more molecules.”

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” are used as equivalents and may be understood to permit standard variation as would be understood by those of ordinary skill in the art; and (v) where ranges are provided, endpoints are included. If a specific, numerical definition of the term is required for the rules of a particular jurisdiction, then the term “about” as used herein refers to plus or minus 10% of a given value.

### EXAMPLES

### Example 1

Gold nanoparticles (AuNP) are of interest in radiotherapy for their ability to radiosensitize tumors. Intra-tumoral distribution of NPs is a factor that greatly impacts the radiation enhancement effects, therefore the development of a suitable AuNP imaging methods in humans is an important step in translational research in gold nanoparticle enhanced radiation therapy. Several imaging techniques have been investigated in the past, including x-ray fluorescence computed tomography (XFCT), micro-CT, dual-energy CT, spectral photon-counting CT, optical spectroscopy, photo-acoustic imaging, surface-enhanced Raman spectroscopy techniques, along with multimodal imaging and labeling techniques for MRI and PET (by ⁶⁴Cu isotope). At this time, however, many of these techniques are still in exploratory phase and are limited to small animal models or shallow regions due to small penetration of signal and small concentration of RCA in tissues

XFCT is a technique that can identify, quantify, and locate elements within objects by detecting characteristic x-rays stimulated by an excitation source. In XFCT of gold, k_{α1} (68.80 keV), k_{α2} (66.99 keV) and K_{β1} (78.0 keV) photons are emitted (fluorescence yield of 0.964) when an electron in the L shell fills a K-shell vacancy created by the photoelectric absorption (PE) of a photon with energy larger than the K-edge (80.72 keV). The XFCT signal must be acquired at different positions and for many projections to allow CT-like reconstruction. Therefore, it requires spectroscopic detectors to identify the characteristic fluorescence peaks (k_{β1,2} in this study) and differentiate it from the Compton background in the spectral plots for each detection angle and/or detector pixel. Furthermore, the collimation of the detectors is necessary to identify the incoming direction of the fluorescence signal. To our knowledge, XFCT has not been applied to imaging of AuNP in humans. Determination of nanoparticle distribution in tissue by means of XFCT has been demonstrated in preclinical models. Application of this imaging method to larger volumes faces several challenges, including large dose from imaging (>70 cGy per slice), large total data acquisition time (>1 h per slice), and attenuation and scatter of k_{α1,2} photons.

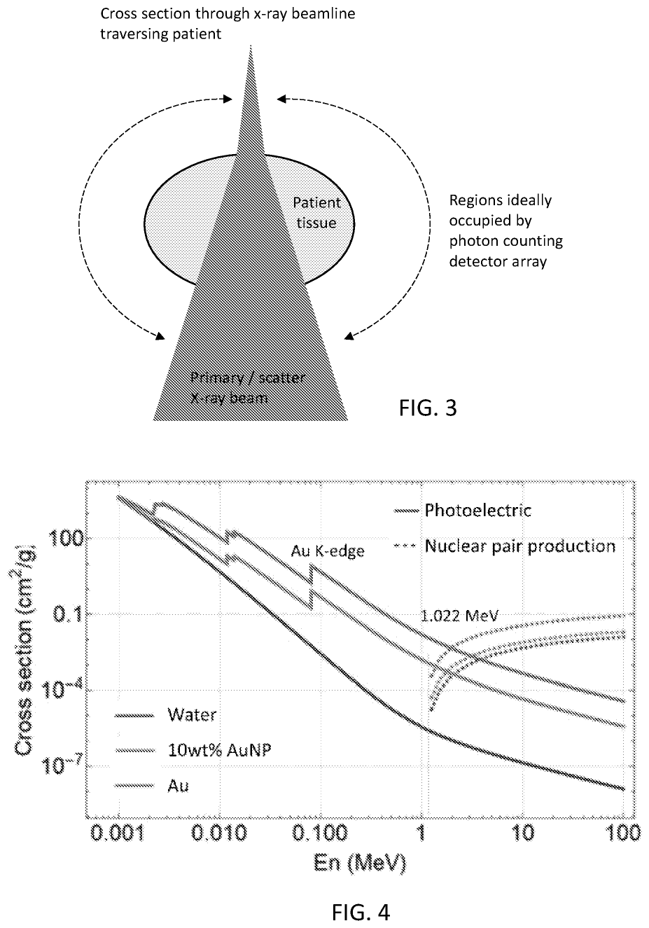

In the standard PET-CT, typically 5-10 mCi or 200-400 MBq ¹⁸F-FDG solution is injected intravenously by a rapid saline drip into a patient who has been fasting for at least 6 h, and who has a suitably low blood sugar. ¹⁸F-FDG, a glucose analog, is preferentially taken up by cancer tissue due to enhanced glycolysis. Positrons directions, which are detected in coincidence by a ring of fast photon counting detector array. PET-CT imaging requires about 20 min scanning time, and the image reconstruction makes use of CT data to correct for attenuation and display anatomical features. PET-CT is one of the most sensitive techniques capable of detecting pico- to nano-molar concentrations in vivo. Noteworthy, when photons above 1.02 MeV are used to image or treat cancer patients, electron-positron pair production (PP) occurs and 511 keV annihilation photon pairs are generated. In materials with high atomic numbers (high Z) the PP cross-sections can be markedly greater than in water, which might be used to quantitatively identify the presence and ascertain the location of nanoparticles in a similar way as in PET imaging. For instance, FIG. 4 shows the cross-sections for PE and for PP in water, gold, and 10 wt % atomic gold-water mixture. Not only the PE cross-section is greater for gold, which is exploited in NP-enhanced radiotherapy, but the PP cross-section can also be several orders of magnitude greater than that in water, which might be exploited in imaging of nanoparticle distribution

We investigated the feasibility of AuNP imaging by detecting 511 keV photons resulting from positron annihilation following PP in gold due to MV irradiation (MVIPE). We compared the MVIPE technique to x-ray fluorescence imaging of k_{α1} and k_{β2} photons resulting from kVp irradiation (XFCT). Normally, the direct detection of 511 keV photons would require a spectroscopic detector to distinguish them from the Compton-scattered radiation. Since the positron annihilation has the unique property of generating a pair of correlated photons with nearly opposite direction and with orthogonal polarization, coincidence counting of the 511 keV pairs may overcome the need of spectroscopic detectors.

### Materials and Methods

Initially, a one-dimensional (1D) geometry was used to acquire the general attenuation and PP properties of the MV beams along the beamline, followed by further evaluation using a 3D water phantom used to obtain angular properties. 1D results were obtained by means of CEPXS/ONEDANT coupled photon-electron radiation transport computer code (Lorence and Morel 199, Jordan 2005) while 3D simulations were performed using MCNP6 Monte Carlo code (Goorley et al 212).

### 1D Deterministic Simulation

CEPXS/ONEDANT is a deterministic radiation transport code that simulates all the processes that are generally considered in Monte Carlo (MC) methods. The cross sections used by CEPXS are essentially the same as those included in the ITS code system (Halbleib et al 1992, Franke et al 2008). ONEDANT solves the Boltzmann equation in 1D in an infinite parallel plane geometry or in spherical symmetry. The code has been extensively tested against MC simulations as well as validated against experiments. For medical physics applications in nanoscopic and microscopic geometries it was shown that it yields the same results as MC simulations but without the significant uncertainties that MC methods entail in such small volumes (Tsiamas et al 2013, Zygmanski et al 2013). We also benchmarked the deterministic results against MCNP6 in the 1D geometry used in this study.

AuNP contrast was distributed in a 5 cm region at 15 cm depth within a 35 cm water phantom. Three AuNP concentrations were investigated: 0.1%, 1% and 10% by weight (wt %), corresponding to 1, 10 and 100 mg of gold per gram of water. This range on nanoparticle concentration has been achieved in preclinical and clinical studies. For instance, concentration values of 37.5 mg g⁻¹ (3.75% wt) were obtained via direct injection into eye, and of 50 mg ml⁻¹ (5% wt) into mice tumor tissues. Bonvalot et al reported the direct NP injection of 53.3 mg ml⁻¹ (5.33% wt) in human soft tissue sarcoma of the limbs (Bonvalot et al 206). The nanoparticle dispersion was assumed to be in homogeneous atomic mixture of gold and water. We considered x-ray sources in the kVp-range (100, 120, 150 kVp) for the x-ray fluorescence study, and in the MV-range for the pair-production study (Co-60, 2 MV, 6 MV, 6MV with closed multi-leaf collimators (MLC), 15MV). In the former we are interested in the characteristic k!1,2 x-rays emitted from gold while in the latter we look for the 511 keV photons generated by positrons annihilation. The kVp spectra were obtained using the Speckcalc software (Poludniowski et al 2009) while the MV spectra were obtained from references (Mora et al 199, Kim et al 2001, Sheikh-Bagheri and Rogers 2002, Tsiamas et al 2014a, 2014b). Specifically, Co-60 therapy beam spectrum was taken from Mora et al (1999), 2 MV and 6 MV were obtained from Tsiamas et al (Tsiamas et al 2014a, 2014b), 6MV with closed MLC was obtained from Kim et al (200) and 15 MV from Bagheri et al (Sheikh-Bagheri and Rogers 2002). Energy deposition and photon, electron and positron fluxes were obtained as function of phantom depth. Fluxes at different planes were computed with energy binning having bin sizes down to 0.1 keV about the gold k_{α1,2} shell energy and 0.5 keV about 511 keV. The net photon flux due to x-ray fluorescence and due to 511 keV annihilation photons within their respective energy bins about the k_{α1,2} and 511 keV lines (hereinafter referred to as Δflux) was obtained by subtracting the Compton-scattered photons from the angular or scalar flux spectra at the detector plane. Mathematically, Φ_{Δ}=Φ_{Total}−Φ_{Compton} in the energy bin about either 511 keV or the k_{α1,2} line, where Φ_{Δ} is Δflux, Φ_{Total} is the total flux in the energy bin of interest and Φ_{Compton} is the background flux of photons in the same energy bin, generated by Compton scattering.

### 3D Monte Carlo Simulation

Monte Carlo simulation in a 3D geometry was performed using the Monte Carlo N-Particle Transport Code System MCNP6.1 (Goorley et al 2012). In this model an x-ray pencil beam (1 cm diameter) is incident on a 3D phantom made of a water cylinder (35 cm diameter, 50 cm length). At its center, a smaller coaxial cylinder (5 cm diameter, 5 cm length) is placed, filled with homogeneously distributed gold, simulating AuNP, in atomic concentrations of 0.1 wt %, 1 wt % and 10 wt %. Two x-ray sources are considered in this study: 150 kVp and 15 MV. The water cylinder is surrounded by an array of 120 detectors in a cylindrical geometry 20 cm from the central axis. Each detector element is 10 cm long and 1 cm wide.

In order to compare the attenuated fluxes that reach the detectors in the XFCT versus the MVIPE techniques, simulations were performed without collimation. We envision MVIPE using coincidence counting without collimation or with partial collimation. Comparison of collimated XFCT and the corresponding MVIPE with smaller detectors is provided in the Discussion section.

The scalar photon flux was computed using the track length estimator (tally type F4) with energy binning, averaged in the volume of each detector. Total energy deposited in the water and AuNP regions were scored using tally *F8 and the dose was calculated by dividing the results by the voxel mass. The number of photons within the narrow energy bins containing the 511 keV and the k_{α1,2} lines were obtained by subtracting the Compton scattered background (in the spectral plot) from the scalar flux spectra at each detector position. The XFCT simulations were run with the 150 kVp spectrum with photon and electron cut-off energies set to 10 keV. The MVIPE simulations were run with the 15 MV spectrum; coupled electron and positron transport was enabled (MODE: P E) down to the electrons cut-off energy of 500 keV. The cut-off energy of photons was 10 keV. Cut-off energies were selected below the relevant energy thresholds of this study, i.e. below the k_{α1,2} fluorescence emission energies in the XFCT study and below the 511 keV annihilation in the MVIPE study. Because in this study we are interested in the annihilation photons, enabling lower cutoff energies would only slightly alter the results by increasing the actual number of 511 keV photons. This observation was made based on simulations with different cutoff energies between 1 and 500 keV. Although the resulting photon spectrum is somewhat different at lower energies, with the choice of 500 keV cutoff energy, the 511 keV pulse height was reduced of about 8%-10% while the Compton background in the relevant energy bin was not affected. The choice was forced by the limited computational power and the signal loss was considered acceptable in a preliminary study. The overall effect of this cut-off energy choice was a significant speed up of the computation at the cost of reduced Δflux of the 511 keV photons. The simulations were run for 10⁹ histories and the flux uncertainties were below 4% in XFCT and below 1% in MVIPE.

### Results

### 1D Deterministic Simulation

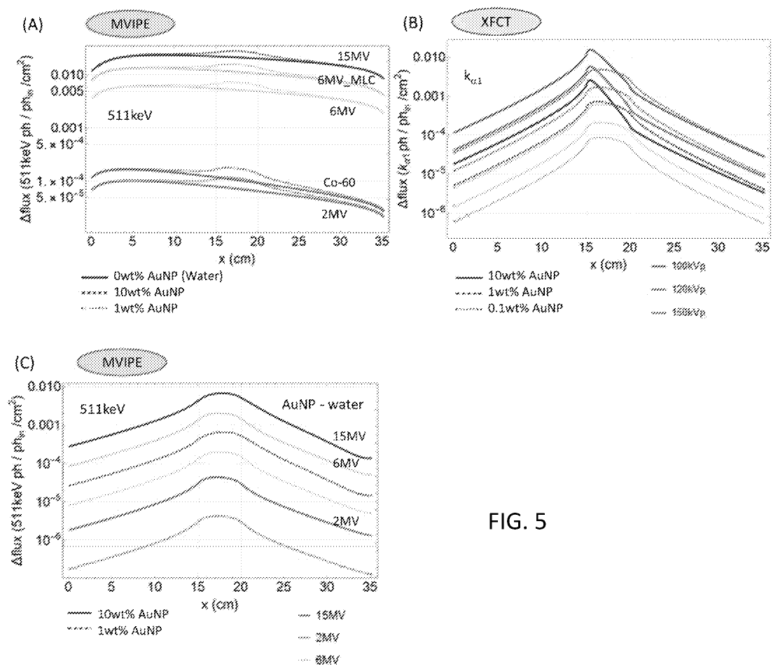

In figure “we report the flux of the spectral lines of interest (511 keV for MVIPE and k_{α1} line for XFCT) after the subtraction of the Compton-scattered photons in the same energy bin (i.e. Δflux), as a function of depth in the water phantom with varying amount of AuNP (0 wt %, 1 wt % and 10 wt %) for MV (FIG. 5(A)) and kVp (FIG. 5(B)) sources. The shape of the curves in FIG. 5(B) reflect the larger absorption of photons in the AuNp filled region at larger concentration. Data are shown normalized per source photon. Note that here k_{α1} photons are characteristic emission of gold only, while 511 keV are annihilation photons generated both by gold and water. Coincidence detection of these photons permits the reconstruction of the location at which they are generated (similar to PET) as will be discussed later. PP in the AuNP region is greater than that in water. The flux difference of the 511 keV photons with and without AuNP is shown in FIG. 5(C). The production rate of k_{α1} photons at the AuNP location per incident source photons is similar to that of the 511 keV photons but their attenuation by water is greater. Using NIST mass-energy absorption coefficients (Hubbell and Seltzer 2004), 15 cm of water attenuates 95% of the k_{α1} photons and 76% of 511 keV photons.

FIG. 4 shows the contribution to Δflux coming from the AuNP region, at phantom the entrance and exit planes for various AuNP concentrations and for different beam energies for both MVIPE and XFCT techniques. The data compare the relative flux values for defined AuNP concentrations, which may be subsequently used for the optimization of the x-ray source spectrum for a given AuNP imaging modality. The flux difference of the 511 keV photons with and without AuNP (i.e. the 511 keV photon flux generated in the water-AuNP mixture less the 511 keV photon flux generated in water only) is obtained at the edge of the AuNP filled region and rescaled to account for the attenuation in 15 cm of water (76%). In this way, we estimate the number of annihilation photons at the detector plane, which is coming from the AuNP. Since Δflux accounts for photons at the 511 keV line we applied attenuation correction only for this energy. We compare the Δflux of the 511 keV photons generated using MV sources with the k_{α1} signal generated using kVp sources. The latter have no angular correlation, and one would require the use of a XFCT pin-hole to limit to a narrow solid angle the detection of the incoming photons originating in a given voxel in the phantom which would greatly reduce these values. The results suggest a linear relationship between Δflux and AuNP concentration up to a concentration of 10% wt in the MVIPE case while the XFCT method shows saturation at large concentration mainly due to attenuation inside the AuNP region.

### 3D Monte Carlo Simulation

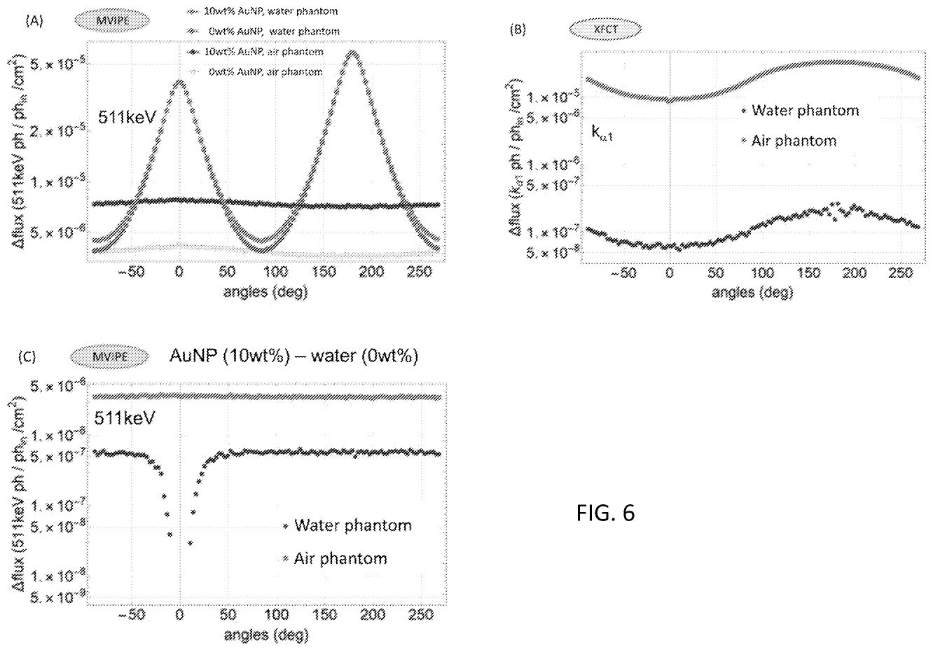

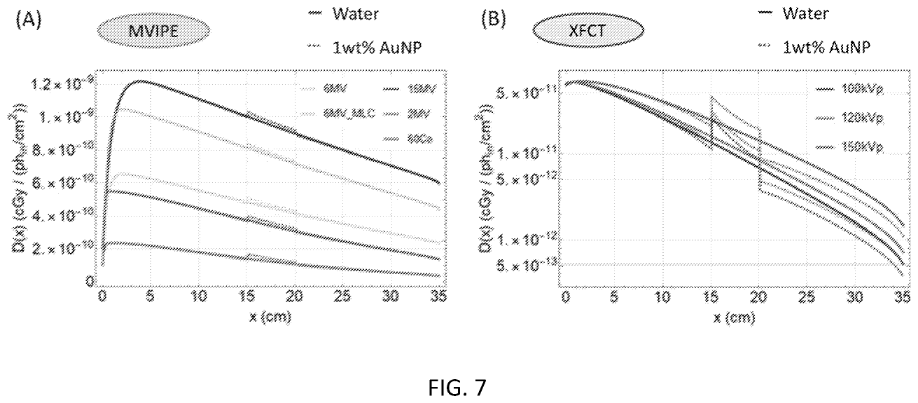

In FIG. 6, Δflux at each detector is plotted as a function of detector angle with respect to the incident beam direction (0°). In FIG. 6(A) we show the cases of 511 keV photons generated by positron annihilation when a 15 MV pencil beam irradiates the cylindrical phantom containing 0 wt % AuNP (water) and 10 wt % AuNP in the central cylinder (5 cm diameter). The large cylindrical phantom (having 35 cm diameter) is filled either with water or air. In FIG. 6(B) we show the k_{α1} photons generated by 150 kVp source interacting with 10 wt % AuNP concentration in the small cylinder for either water-filled or air-filled large phantom. FIG. 5(C) shows the 511 keV photons from AuNP only, after subtracting the contribution to the annihilation photons generated by PP in water. Uncertainties include MC and error propagation after Compton background subtraction; the MC uncertainties are lower than 1% (4%) for MVIPE (XFCT) when both simulations were run for 10′ histories. The use of an air-filled phantom highlights the effect of 511 keV photons generated by PP in water (compare curves for water versus air phantom in FIG. 6(A)) and the effect of attenuation of the characteristic photons for both the XFCT and MVIPE techniques (see FIGS. 6(B) and 6(C)).

### Positron Range

The positron range is the distance traveled by the positron before the annihilation occurs and it increases with the positron energy. This range contributes to blurring and reduces spatial resolution. We show the positron flux/cm²/source photons as a function of depth in water obtained from the deterministic 1D simulation for different MV sources and with AuNP concentration of 0 wt % (water only), 1 wt % and 10 wt %. We notice that increasing the beam energy broadens the energy distribution of the positron flux downstream the AuNP region. For instance, in the case of Co-60 and 2 MV sources the positron flux for the 10 wt % AuNP case have a sharp edge at the interfaces (at 15 and 20 cm depth) while in the 15 MV case the position flux extends outside the interface in the downstream direction of the beam. We plotted the normalized energy spectra of the positrons at the distal AuNP edge (at 20 cm depth) for different MV photon sources. Using these spectra and the positron range in water obtained from NIST (Berger et al 20 5) we calculated the mean positron ranges shown in Table 1. It is seen that the 15 MV source generates positrons whose mean range in water is 13 mm while a 6MV source (with closed MLC) gives rise to positrons with a range of 5.5 mm:

TABLE 1
mean positron energy and range for
the megavoltage sources considered

| | Mean | Mean |
|---|---|---|
| d) | Energy (MeV) | Range (cm) |
| 15 MV | 2.64 | 1.3 |
| 6 MV MLC | 1.19 | 0.55 |
| 6 MV | 1.29 | 0.60 |
| 2 MV | 0.38 | 0.13 |
| Co-60 | 0.14 | 0.03 |

We must also consider that in the present study we simulated AuNP as a mixture of gold and water molecules, and we computed the positron range in water. In solid metals, like gold, the positron lifetime is much shorter than in water (116 ps in gold versus 1.84 ns in water at 20° C.) since the electron density is higher. Therefore, in reality, the positrons generated and traveling in the bulk AuNP will lose large part of their energy inside of it, their range would be shorter than illustrated in Table 1, and they would annihilate much closer to the nanoparticle.

### Deposited Dose

FIG. 7 shows the dose deposited as a function of depth in the phantom with and without a 1 wt % AuNP region for MV sources (FIG. 7(A)) and kVp sources (FIG. 7(B)). Dose is reported in cGy normalized by the number of source photons incident on 1 cm² of phantom surface. Dose deposited to the phantom by most MV sources per incident x-ray is more than 1 order of magnitude higher than that when using kVp sources. The dose enhancement ratio in the target volume for 1 wt % AuNP mixture is about 1.04 for 6MV beam and it is 1.5 for 150 kVp beam.

### Discussion

### The Detectable Number of Photons and the Resulting Spatial Resolution

The fluxes shown in FIG. 6 are the average number of annihilation or k_{α1} photons reaching each detector, normalized to one incident source photon/cm². The use of a 2.5 mm pin-hole, similar to the one used in previous XFCT studies would reduce the detected flux by a factor of about 2×10³, because of both smaller detector area (0.05 cm²) and smaller solid angle of collection (from an area of 0.05 cm² rather than from 5 cm²). Therefore, by using such pinhole in the simulation shown in FIG. 6(B), a detector at 90° would receive about 0.05 photons out of 10⁹ incident source photons for a AuNP concentration of 10 wt %. By considering the approximate linear dependence with AuNP concentration we estimate that the flux would be about 0.005 photons/AuNP wt %/10⁹ source photons. Detection efficiency would further reduce the counts; the XR-100T-CdTe gamma and x-ray spectrometer detector (Amptek Inc., Bedford, MA) used by Manohar et al (Manohar et al 2016) has a quantum efficiency of 0.9 at the k_{α1,2} energies. In their small animal setup, Manohar obtained about 500 counts above Compton background at the k_{α1} peak in 15 s of acquisition at a dose rate of 0.826 cGy min⁻¹ with a concentration of about 5 wt % (see FIG. 5 in the paper Manohar et al 2016). With the same detection system and a large phantom, one would need about 10⁴ times longer acquisition time or much larger dose.

The standard XFCT technique is therefore not suitable for AuNP imaging inside large objects (patient) for the following reasons: (1) large attenuation of the k_{α} photons in water/tissue (95% in 15 cm of water); (2) isotropic emission (1/R² geometric attenuation); (3) small pinhole needed to identify the direction of the photon and perform image reconstruction.

In contrast, the MVIPE technique could employ a PET-like reconstruction technique, which exploits coincidence detection of the 511 keV annihilation photon pairs, and it does not require the use of a pin-hole collimator to identify the direction and reconstruct the image. The emission of 511 keV photons is isotropic but each photon pair is emitted at nearly 180°. Further, the attenuation of 511 keV photons in water/tissue is smaller than that of the k-photons in XFCT (76% in 15 cm of water). The spatial resolution of PET scanners, tough, is influenced by several factors such as the size of the detectors, the positron range, the non-perfect collinearity, partial volume effects, the localization of the detector when block detectors are used, and the reconstruction method. The intrinsic resolution is related to the detector size, D, and it is normally given by D/2 at the scanner axis and D at the detector position.

The new PET detectors, such as Compton detectors (Peng et al 2019), would increase efficiency (wide angle whole body) and resolution. Compton Cameras use the kinematics of Compton scattering to deduce the initial photon trajectory and hence, the source region (Todd et al 1974, Everett et al 1977). McNamara et al (2014) and Toghyani et al (20 6) showed that the angular correlation of Compton scattered annihilation photon pairs can serve as a practical discriminatory tool for identification of true coincidences in PET. In our simulation we used 120 detectors positioned on a 40 cm diameter ring, therefore D=1.05 cm. The spatial resolution at the scanner axis is therefore about 5 mm. Using smaller detectors can reduce this. For instance, by means of 5×5 mm² detectors the intrinsic resolution at the axis would be 2.5 mm (similar to XFCT pin-hole collimator described above). The non-collinearity occurs because the 511 keV annihilation photons are not emitted at exactly at 180° due to a small residual momentum of the positron at the end of its range. The maximum deviation from the 180° direction is about ±0.25°. The contribution from non-collinearity worsens with larger diameter of the detectors ring, and it amounts to 1.8-2 mm for currently available 80-90 cm PET scanners (0.9 mm for the 40 cm diameter scanner considered in this work). All these factors must be summed up in quadrature to estimate the effective spatial resolution. Commercial PET scanners have about 5 mm spatial resolution at 1 cm from the scanner axis.

As shown in FIG. 6(A), when 10 wt % AuNP is used in 15 MV MVIPE, at 90° a detector receives about 4000 photons/cm² per 10⁹ source photons from water and an additional ˜500 photons cm⁻² per 10⁹ source photons from AuNP. To obtain an intrinsic spatial resolution similar to the XFCT case (2.5 mm) we would need to reduce the detector size to 5×5 mm². Assuming that each detector has an efficiency at 511 keV (coincidence detection efficiency of 0.1×0.1=0.01), we would count about 1.25 coincidences/10⁹ source photons coming from AuNP. By considering the linear relation of the Δflux with AuNP concentration below 10% wt, we estimate about 0.125 coincidence counts/AuNP wt %/10⁹ source photons, which is about 25 times larger than that for XFCT.

Note that kVp irradiators used for small animal studies typically produce fluxes of about 10⁸-10⁹ photons cm⁻² mAs⁻¹ at 100 cm while clinical Linac produces fluxes of about 10-10⁸ photons/cm²/pulse at the same distance from the source. Therefore delivering 10⁹ photons requires 1-10 mAs in kVp irradiators and 10-100 linac pulses.

In XFCT kilovoltage energies are used. The scanning time for experimental XFCT systems is relatively large even for small objects, and this equipment is not developed for patients. The MVIPE technique employs megavoltage (MV) beams. Total body PET has been developed and linac-PET is under development at RefleXion Medical Inc. This indicates a potential advantage of MVIPE over XFCT for real patients. The scanning time for experimental XFCT systems is relatively large even for small objects, and this equipment is not developed for patients. The MVIPE technique employs MV beams. Note the new PET technology such as Total Body PET has been already developed and linac-PET is under development at RefleXion Medical Inc (Fan et al 20 2). This indicates a potential advantage of MVIPE over XFCT for imaging nanoparticle distribution inside patients during treatment.

### Additional Considerations

Excessive Compton background and PP in water/tissue may be a limiting factor to a practical implementation of the MVIPE imaging technique. Nevertheless, since the rate of PP and the Compton background in air is much smaller than in water or in tissue, the MVIPE technique may be more suitable for AuNP imaging in lung tumors rather than in deeply seated tumors (pelvis). Furthermore, the attenuation of 511 keV photons in lung is much smaller than in tissues.

### CONCLUSIONS

We investigated the possibility to detect gold nanoparticles by means of a novel MV x-ray induced positron emission (MVIPE) and we compared it to standard XFCT. We conclude that:
MVIPE technique using a 15 MV pencil beam and 10 wt % AuNP detects about 4.5×10³ 511 keV-photons cm⁻² at 90° w/r to the incident beam per 10⁹ source photons/cm²; 500 of these come from AuNP. In contrast, the XFCT technique using 150 kVp detects only about 100 k_{α1}-photons cm⁻² per 10⁹ source photons cm⁻²
MVIPE coincidence counting has the advantage of providing the directionality and PET-like spatial reconstruction with attenuation correction, while image reconstruction/directionality information in XFCT requires the use of a pinhole, which drastically reduce the count rate.
MVIPE is about 25 times more sensitive than XFCT for similar intrinsic spatial resolution (about 2.5 mm) when uncollimated 5×5 mm² detectors in MVIPE are compared with collimated XFCT with 2.5 mm diameter pinhole and considering detector efficiency of 10% at 511 keV and 90% at k_{α1}.
MVIPE annihilation photons originate both from Au and from water while in XFCT k_{α1,2} peaks are characteristic of Au only.
XFCT k_{α1,2} photons are generated locally at the AuNP location while in the MVIPE technique the positrons have a finite range which depends on their energy. In realistic AuNP (i.e. not in atomic mixture with water) the positron lifetime is much shorter than in water and therefore the positron range would be shorter as well, reducing range blurring effects in the image.
For MVIPE the optimal MV source is 15 MV in terms of yield but in terms of positron range the lower the energy the better. 6 MV spectra generated with closed MLC is a good compromise since it provides a similar yield to the 15 MV source while keeping the average positron range at about 5.5 mmin water. This could be used for imaging session only without therapeutic intensity beam.
The number of photons scales linearly with AuNP concentration in MVIPE up to 10% wt, while in XFCT there is a saturation at high concentrations due to the strong attenuation of the source beam in the AuNP region.
In regular PET equipment, there is no Compton continuum across the 511 keV annihilation photons that reach the detector. In MVIPE there is a higher background radiation due to Compton scattering of the MV source photons, which must be suppressed in measurements or in data processing. The MVIPE technique we described in this paper is a promising technique for imaging of AuNP even in large patients. The findings in this study warrant further investigation.

One specific detector arrangement is described in the provisional application filing to which this application claims priority, namely, U.S. Provisional Patent Application No. 63/225,415, filed Jul. 23, 2021, which is incorporated herein in its entirety by reference.

1. A system for detecting and imaging high Z agents within anatomy of a subject, the system comprising:
a therapeutic medical high energy photon source adapted to provide megavoltage ionizing radiation with an emitted photon energy of no lower than 1.02 MeV;
a fast photon counting detector array positioned so that at least two pixels are oriented to simultaneously measure two coincident, 511 keV photons with opposing directions originating from a location;
a processor; and
a memory having stored thereon instructions that, when executed by the processor, cause the processor to:
a) initiate delivery of the megavoltage ionizing radiation from the therapeutic medical high energy photon source to the location;
b) initiate detection of the two coincident photons with opposing directions originating from the location;
c) determine photon trajectories of the two coincident photons with opposing directions, thereby providing a source location for the two coincident photons where the photon trajectories meet; and
d) generate a report including a high Z agent position within the anatomy of the subject derived from the trajectories of the two coincident photons with opposing directions within the subject's anatomy,
wherein the high Z agent has an atomic number of greater than 7.4 and/or a density that is greater than surrounding tissue within the anatomy.

2. The system of claim 1, wherein the at least two pixels is a plurality of pixels and pairs of the plurality of pixels are oriented to simultaneously measure two coincident, 511 keV photons with opposing directions originating from a number of locations that corresponds to the number of pairs.

3. The system of claim 2, wherein the report includes multiple high Z agent positions.

4. The system of claim 3, wherein the instructions, when executed by the processor, further cause the processor to generate an image including the locations and concentrations of the high Z agents within the anatomy of the subject.

5. A method of detecting and imaging high Z agents within anatomy of a subject, the method comprising:
a) delivering megavoltage ionizing radiation from a therapeutic medical high energy photon source to at least one high Z agent located within the anatomy of the subject;
b) subsequently, detecting two coincident photons with opposing directions originating from the high Z agent with a fast photon counting detector array positioned so that at least two pixels are oriented to simultaneously measure the coincident photons with opposing directions;
c) determining photon trajectories of the detected two coincident photons, thereby providing a source location for the two coincident photons where the photon trajectories meet; and
d) generating a report including a high Z agent position within the anatomy of the subject derived from the trajectories of the two coincident photons,
wherein the high Z agent has an atomic number of greater than 7.4 and/or a density that is greater than surrounding tissue within the anatomy.

6. The method of claim 5, wherein the detecting of step b) comprises detecting a plurality of pairs of coincident photons with opposing directions originating from a plurality of high Z agents with the fast photon counting detector array, wherein the determining photon trajectories of step c) comprises determining photon trajectories from the detected plurality of pairs of coincident photons, thereby providing a plurality of source locations for the plurality of pairs of coincident photons where respective source trajectories meet.

7. The method of claim 5, wherein the report is an image of the anatomy of the subject.

8. The method of claim 7, wherein the image includes a concentration distribution of the high Z agents.

9. The method of claim 5, wherein steps a), b), and c) are repeated at different orientations relative to the anatomy of the subject to produce a 3-dimensional image.

10. The method of claim 5, wherein the subject is dosed with a radiosensitization/contrast agent (RCA).

11. (canceled)

12. The system of claim 1, wherein the high Z agent has an atomic number of greater than 7.4.

13. The method of claim 5, wherein the high Z agent has an atomic number of greater than 53.

14. (canceled)

15. (canceled)

16. The system of claim 1, wherein the high Z agent is a nanoparticle.

17. (canceled)

18. (canceled)

19. The method of claim 5, wherein the high Z agent is present in the tissue in an amount by weight of between 0.10% and 10%.

20. (canceled)

21. The system of claim 1, wherein the photons from the therapeutic medical high energy photon source has a beam energy of between 2 MV and 30 MV.

22. The system of claim 1, wherein the coincident photons are from a 6 MV beam and are generated with closed multileaf collimators.

23. (canceled)

24. (canceled)

25. (canceled)

26. The method of claim 10, wherein the two coincident photons with opposing direction are used to discriminate events that originate from the subject's tissue alone and are signatures of RCA dose distribution inside the subject.

27. The method of claim 10, wherein the two coincident photons with opposing direction are used to discriminate events that originate from RCA alone and are signatures of concentration of RCA in tissue.

28. The system of claim 1, wherein the fast photon counting detector array discriminates between photons arising from positron annihilation and photons arising from Compton scattering by their temporal, spatio-angular, and spectral distribution.

29. The system of claim 1, wherein the fast photon counting detector array comprise a Compton detector.

30. (canceled)

31. (canceled)

32. (canceled)

33. (canceled)

34. (canceled)
